(19) United States
(12) Patent Application Publication
(10) Pub. No.: US 2026/0137850 A1
(43) Pub. Date: May 21, 2026

(54) TARGET LIQUID TREATMENT APPARATUS

(71) Applicant: PHYSIOLOGAS TECHNOLOGIES, INC., Sagamihara-shi, Kanagawa (JP)

(72) Inventors: Kazuyoshi MIYAWAKI, Sagamihara-shi, Kanagawa (JP); Kenichi KOKUBO, Sagamihara-shi, Kanagawa (JP); Hiroyuki AOKI, Sagamihara-shi, Kanagawa (JP)

(21) Appl. No.: 18/878,319

(22) PCT Filed: Aug. 18, 2023
(86) PCT No.: PCT/JP2023/029885

§ 371 Date: Dec. 23, 2024

(30) Foreign Application Priority Data
Aug. 23, 2022 (JP) 2022-132837

(51) Int. Cl. A61M 1/16; A61M 1/34

(52) U.S. Cl. CPC A61M 1/1603; A61M 1/3413; A61M 2205/3334; A61M 2205/3337; A61M 2230/20

(57) ABSTRACT

A concentration of a substance to be removed in target liquid on an upstream side of a joining point of a second flow path in a first flow path is measured as a first concentration C1 based on an output signal of a first concentration sensor. A concentration of the substance to be removed in target liquid on a downstream side of the joining point of the second flow path in the first flow path is measured as a second concentration C2 based on an output signal of a second concentration sensor. When a difference ΔC between the first concentration C1 and the second concentration C2 is less than a concentration difference threshold, at least one of decreasing degree of opening of a first flow rate control valve and increasing degree of opening of a second flow rate control valve is executed.

### TECHNICAL FIELD

The present invention relates to a technique for appropriately removing toxins such as potassium ions from dialysis effluent and the like during dialysis treatment of patients with chronic renal failure and the like.

### BACKGROUND ART

In hemodialysis, potassium ions and the like are removed by diffusion based on the difference in concentration between blood and dialysate in a dialysis machine by bringing the blood and the dialysate into contact with each other through a dialysis membrane. The concentration of dialysate components entering the dialysis machine is predetermined, and the dialysis effluent to which potassium and other toxins have been transferred from the blood is directly discarded.

While the amount of removed potassium ions and the concentration of potassium ions in the blood after dialysis will vary with blood flow rate and dialysis time, since these are treatment conditions that are primarily determined by other factors, the potassium concentration in the dialysate is generally varied in order to attain an appropriate amount of removed potassium ions and an appropriate concentration of potassium ions in the blood after dialysis. For this reason, several dialysates with different concentrations are commercially available.

A large difference between the potassium concentration in the dialysate and the concentration in the patient's serum has been reported to increase the incidence of arrhythmias and the risk of hospitalization and death (refer to Non Patent Literature 1). Therefore, treatment preferable to the patient can be realized when the potassium concentration can be varied so that the difference between the concentration in the patient's serum and the concentration in the dialysate is reduced. In consideration thereof, a method has been proposed in which the potassium concentration in the dialysate at the Start of treatment is set 1.5 mEq/L lower than that in the patient's serum and the potassium concentration in the dialysate is then exponentially reduced during treatment such that the difference between the potassium concentration in the dialysate and the concentration in the patient's serum decreases (refer to Non Patent Literature 2). However, since there is no apparatus for measuring the concentration in blood during dialysis and feeding back the results in order to control changes in the concentration of the dialysate, a method of treating patients while varying the concentration of dialysate to an appropriate level has not been realized.

While adsorbents that remove potassium ions include ion exchange resins (potassium adsorption and removal filters) that remove potassium ions from blood for transfusion, they are not used for adsorption of dialysis effluent (refer to Patent Literature 1).

### CITATION LIST

### Patent Literature

Patent Literature 1: Japanese Patent Laid-Open No. 2001-161807

### Non Patent Literature

Non Patent Literature 1: Steven M. Brunelli1, David M. Spiegel, Charles Du Mond, Nina Oestreicher, Wolfgang C. Winkelmayer and Csaba P. Kovesdy. Serum-to-dialysate potassium gradient and its association with short-term outcomes in hemodialysis patients. Nephrol Dial Transplant (2018) 33:1207-1214
Non Patent Literature 2: Redaelli B, Locatelli F, Limido D et al. Effect of a new model of hemodialysis potassium removal on the control of ventricular arrhythmias. Kidney Int (1996) 50:609-617

### SUMMARY OF INVENTION

### Technical Problem

In currently used dialysis methods in which dialysates are used on a single-pass basis, an amount of potassium removal and post-dialysis potassium concentration are adjusted solely based on dialysate concentration. However, depending on the patient, phenomena such as the following may sometimes occur when dialysis is solely based on dialysate concentration:
(1) Potassium concentration does not drop sufficiently;
(2) Potassium concentration drops too low; or
(3) The difference between the patient's blood concentration and the concentration in the dialysate becomes too large particularly at the beginning of dialysis.

Adopting a method of treating and reusing a dialysis effluent, a hemodiafiltrate, or a hemodiafiltration fluid coming out of a dialyzer or a diafilter (dialysate recirculation dialysis) makes it easier to adjust the concentration difference between potassium ion and other urine toxins in the dialysate so that it does not become too large. However, even in such cases, the potassium concentration cannot be prevented from dropping too low and the difference in concentration between the blood and the dialysate cannot be properly controlled.

In consideration thereof, an object of the present invention is to provide an apparatus that can efficiently remove substances to be removed such as unwanted ions including potassium ion from a target liquid such as dialysis effluent or the like while maintaining the fluidity of the dialysis effluent or the like and controlling a difference in concentration to an appropriate level.

### Solution to Problem

A target liquid treatment apparatus according to the present invention includes:
a first flow path through which a dialysis effluent, a hemodiafiltrate, or a hemodiafiltration fluid flowing out of a dialyzer or a diafilter flows as a target liquid;
a second flow path which joins the first flow path after branching from the first flow path;
a target substance removal apparatus which is provided in the second flow path and which is capable of selectively removing a substance to be removed contained in the target liquid;
a first flow rate control valve which is provided in the first flow path between a branching point and a joining point of the second flow path;
a second flow rate control valve which is provided in the second flow path;
a first concentration sensor which measures a concentration of the substance to be removed in the target liquid as a first concentration on an upstream side of the joining point of the second flow path in the first flow path;
a second concentration sensor which measures a concentration of the substance to be removed in the target liquid as a second concentration on a downstream side of the joining point of the second flow path in the first flow path; and
a control apparatus for adjusting respective degrees of opening of the first flow rate control valve and the second flow rate control valve based on measured results of the first concentration and the second concentration, wherein
the control apparatus is configured to
execute, when the difference between the first concentration and the second concentration is less than a concentration difference threshold, at least one of decreasing the degree of opening of the first flow rate control valve and increasing the degree of opening of the second flow rate control valve.

### BRIEF DESCRIPTION OF DRAWINGS

FIG. 1 is an explanatory configuration diagram of a target liquid treatment apparatus as an embodiment of the present invention.

FIG. 2 is a first explanatory function diagram of the target liquid treatment apparatus as an embodiment of the present invention.

FIG. 3 is a second explanatory function diagram of the target liquid treatment apparatus as an embodiment of the present invention.

FIG. 4 is a third explanatory function diagram of the target liquid treatment apparatus as an embodiment of the present invention.

### DESCRIPTION OF EMBODIMENT

### (Configuration of Target Liquid Treatment Apparatus)

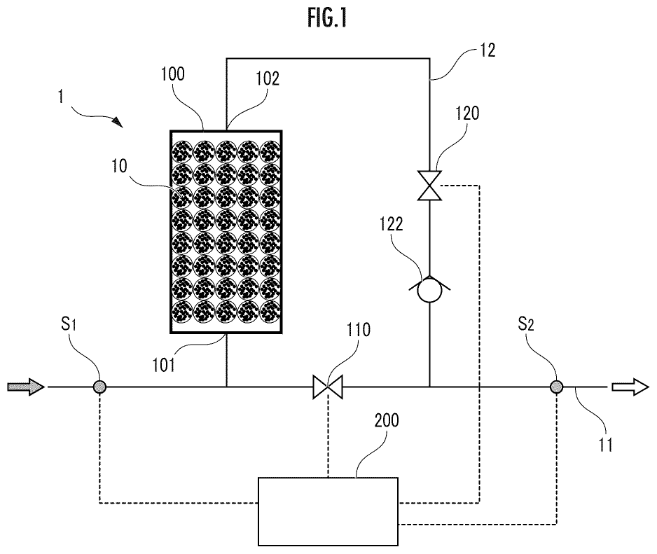

A target liquid treatment apparatus as an embodiment of the present invention shown in FIG. 1 has a first flow path 11, a second flow path 12, a target substance removal apparatus 1, a first flow rate control valve 110, a second flow rate control valve 120, a first check valve 112, a second check valve 122, a first concentration sensor S₁, a second concentration sensor S₂, and a control apparatus 200.

The first flow path 11 is configured so that a dialysis effluent, a hemodiafiltrate, or a hemodiafiltration fluid flowing out of a dialyzer or a diafilter (not illustrated) flows through (or circulates in) the first flow path 11 as a target liquid. The dialyzer or the like is used in dialysis treatment for patients with chronic renal failure, acute renal failure, or those requiring plasma exchange therapy. The target liquid treatment apparatus may be used to reduce potassium levels or to adjust ion balance in blood for transfusion. The second flow path 12 is configured to branch from the first flow path 11 and subsequently join the first flow path 11 on a downstream side of the branching point.

The target substance removal apparatus 1 is provided in the second flow path 12 and has a function of selectively removing a substance to be removed that is contained in the target liquid. The target substance removal apparatus 1 is constituted of a housing 100 and a plurality of ion adsorbents 10 filled or arranged in an internal space of the housing 100. The housing 100 has an inflow port 101 for allowing the target liquid flowing in the second flow path 12 to enter the internal space and an outflow port 102 for allowing a dialysis effluent and the like to flow out of the internal space into the second flow path 12. The target substance removal apparatus 1 is configured to be attachable/detachable and thus interchangeable with respect to a tubular member constituting the second flow path 12.

Each ion adsorbent 10 may contain at least one of an ion exchange resin, a polymer gel, and a ceramic-based adsorbent. For example, the ion adsorbent 10 may be constituted of a base material made of a porous polymer and a large number of ion adsorbing particles dispersed and arranged in the base material. The materials may be capable of selectively adsorbing target substances such as ions including potassium ions, ammonium ions, calcium ions, and/or magnesium ions and/or urinary toxins.

The first flow rate control valve 110 is provided in the first flow path 11 between a branching point and a joining point of the second flow path 12. The second flow rate control valve 120 is provided in the second flow path 12 on a downstream side of the target substance removal apparatus 1. The second flow rate control valve 120 may be provided in the second flow path 12 on an upstream side of the target substance removal apparatus 1. The first check valve 112 is provided in the first flow path 11 on a downstream side of the first flow rate control valve 110. The second check valve 122 is provided in the second flow path 12 on a downstream side of the second flow rate control valve 120. The first check valve 112 and/or the second check valve 122 may be omitted.

The first concentration sensor S₁ is configured to output a signal according to a first concentration C₁ being a concentration of the substance to be removed in the target liquid on an upstream side of the joining point (in the present embodiment, also on an upstream side of the branching point) of the second flow path 12 in the first flow path 11. The second concentration sensor S₂ is configured to output a signal according to a second concentration C₂ being a concentration of the substance to be removed in the target liquid on a downstream side of the joining point of the second flow path 12 in the first flow path 11.

The control apparatus 200 is constituted of a processor (arithmetic processing unit), a memory (storage device) such as a ROM or a RAM, an I/F circuit, and the like. Tasks such as flow rate control processing to be described later are executed as the arithmetic processing unit constituting the control apparatus 200 reads necessary data and programs (software) from the storage device and executes arithmetic processing according to the program with respect to the data. The control apparatus 200 is configured to adjust a degree of opening of each of the first flow rate control valve 110 and the second flow rate control valve 120 based on measurement results of the first concentration C₁ and the second concentration C₂.

### (Functions of Target Liquid Treatment Apparatus)

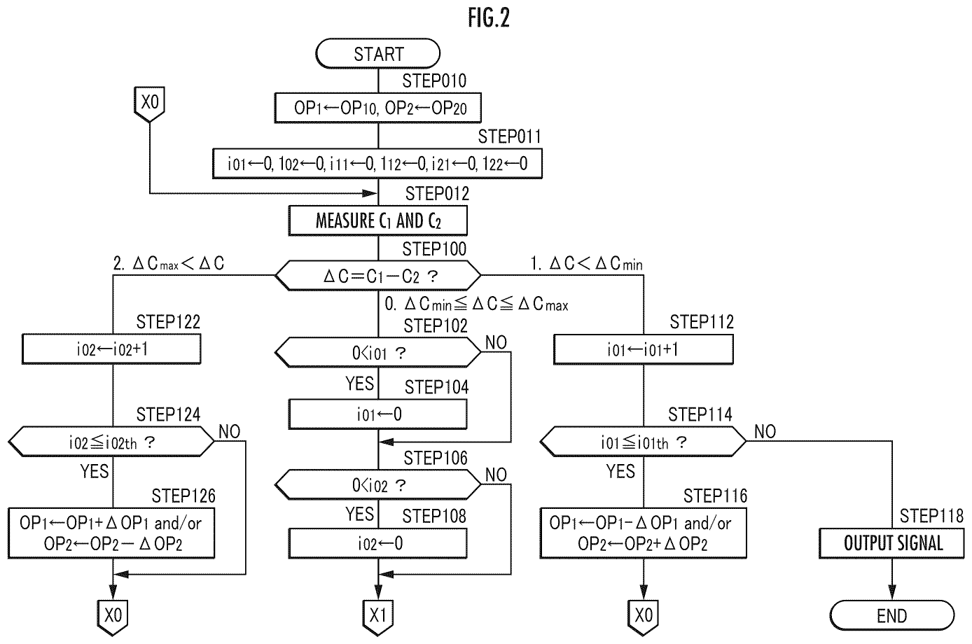

The functions of the target liquid treatment apparatus configured as described above and thus processing procedures by the control apparatus 200 will be described according to the flowcharts in FIGS. 2 to 4.

First, when an operation of the target liquid treatment apparatus is started in response to an ON operation of an operation switch or the like, a degree of opening OP₁ of the first flow rate control valve 110 is controlled to OP₁₀ and a degree of opening OP₂ of the second flow rate control valve 120 is controlled to OP₂₀ (FIG. 2/STEP 010). In addition, each of the difference indexes “i₀₁”, “i₀₂”, “i₁₁”, “i₁₂”, “i₂₁” and “i₂₂” described below is reset to “0” (FIG. 2/STEP 011).

Furthermore, based on an output signal of the first concentration sensor St, the first concentration C; being a concentration of the substance to be removed in the target liquid on an upstream side of the branching point of the second flow path 12 in the first flow path 11 is measured (FIG. 2/STEP 012). In a similar manner, based on an output signal of the second concentration sensor S₂, the second concentration C₂ being a concentration of the substance to be removed in the target liquid on a downstream side of the joining point of the second flow path 12 in the first flow path 11 is measured (FIG. 2/STEP 012).

Next, a determination is made as to whether a concentration difference ΔC=C₁−C₂ being a difference between the first concentration C₁ and the second concentration C₂ is included in a designated concentration difference range [ΔCₘᵢₙ, ΔCₘₐₓ] or is less than a lower limit value ΔCₘᵢₙ or exceeds an upper limit value ΔCₘₐₓ thereof (FIG. 2/STEP 100). Taking into account the distance or time the target liquid flows from the first concentration sensor S₁ to the second concentration sensor S₂, a difference ΔC=C₁(k−m)−C₂(k) between the second concentration C₂(k) at a time point t=k (where k represents a control cycle) and the first concentration C₁(k−m) at a time point t=k−m, which is in the past of time point t, may be used as a basis for the determination processing.

A magnitude of the concentration difference ΔC represents a high or low removal performance (or adsorption performance) of the substance to be removed by the target liquid treatment apparatus. For example, consider a case where a Cv value representing an intrinsic flow rate characteristic according to the degree of opening OP₁ of the first flow rate control valve 110 is Cv₁=f₁(OP₁) and a Cv value representing the intrinsic flow rate characteristic according to the degree of opening OP₂ of the second flow rate control valve 120 is Cv₂=f₂(OP₂). In this case, based on a flow rate Q₁ of the target liquid flowing into the first flow path 11 and a flow rate Q₂ of the target liquid flowing into the second flow path 12, a concentration change amount ΔC₀ in the second flow path 12 upstream and downstream of the target liquid treatment apparatus is derived according to a relational expression (01). A determination of whether or not the concentration change amount ΔC₀ is included in the designated concentration difference range may be made in place of the concentration difference ΔC. Assume that while specific gravity G of the target liquid is the same, differential pressure Δp₁ upstream and downstream of the first flow rate control valve 110 and differential pressure Δp₂ upstream and downstream of the second flow rate control valve 120 are different.

$$Δ {C}_{0} = Δ C ( {Q}_{1} / {Q}_{2} ) = Δ C ( {Cv}_{1} / {Cv}_{2} ) {( Δ {p}_{1} / Δ {p}_{2} )}^{1 / 2} . \qquad ( 01 )$$

When the concentration difference ΔC is determined to be less than the lower limit value ΔCₘᵢₙ of the designated concentration difference range (FIG. 2/STEP 100 . . . 1), the first difference index i₀₁ is increased by “1” (FIG. 2/STEP 112). The first difference index “i₀₁” is an index representing a duration of a situation in which the concentration difference ΔC is less than the lower limit value ΔCₘᵢₙ of the designated concentration difference range. Adjustment may be made for an amount of increase in the first difference index i₀₁ so that the smaller a rate of increase (dΔC/dt) of the concentration difference ΔC, the larger the amount (for example, significantly more than “1” such as “2” or “3”). Next, a determination is made as to whether the first difference index i₀₁ is equal to or lower than a first difference threshold i₀₁ₜₕ (FIG. 2/STEP 114).

When the determination result is positive (FIG. 2/STEP 114 . . . YES), the operation of the first flow rate control valve 110 and/or the second flow rate control valve 120 is controlled so that the degree of opening OP₁ of the first flow rate control valve 110 is decreased by ΔOP₁ and/or the degree of opening OP₂ of the second flow rate control valve 120 is increased by ΔOP₂ (FIG. 2/STEP 116). Accordingly, a flow rate of the target liquid in the second flow path 12 and thus in the target substance removal apparatus 1 is increased. Then, processing of connector X0 and thereafter is executed. In other words, processing of measuring the first concentration C₁ and the second concentration C₂ (FIG. 2/STEP 012) and thereafter is repeated.

Adjustment may be made for the degree-of-opening decrease amount ΔOP₁ of the first flow rate control valve 110 and/or the degree-of-opening increase amount ΔOP₂ of the second flow rate control valve 120 so that the smaller the rate of increase (dΔC/dt) of the concentration difference ΔC, the larger the amount.

On the other hand, when the determination result is negative (FIG. 2/STEP 114 . . . NO), a signal indicating that adsorption by the target substance removal apparatus 1 has reached an upper limit is output through an output interface capable of wired communication or wireless communication with the control apparatus 200 in a form that the user can visually and/or audibly recognize (FIG. 2/STEP 118). Subsequently, the series of processing ends.

When the concentration difference ΔC is determined to exceed the upper limit value ΔCₘₐₓ of the designated concentration difference range (FIG. 2/STEP 0100 . . . 2), the second difference index i₀₂ is increased by “1” (FIG. 2/STEP 122). The second difference index “i₀₂” is an index representing a duration of a situation in which the concentration difference ΔC exceeds the upper limit value ΔCₘₐₓ of the designated concentration difference range. Adjustment may be made for an amount of increase in the second difference index i₀₂ so that the smaller a rate of decrease (dΔC/dt) of the concentration difference ΔC, the larger the amount (for example, significantly more than “1” such as “2” or “3”). Next, a determination is made as to whether the second difference index i₀₂ is equal to or lower than a second difference threshold i₀₂ₜₕ (FIG. 2/STEP 124).

When the determination result is positive (FIG. 2/STEP 124 . . . YES), the operation of the first flow rate control valve 110 and/or the second flow rate control valve 120 is controlled so that the degree of opening OP₁ of the first flow rate control valve 110 is increased by ΔOP₁ and/or the degree of opening OP₂ of the second flow rate control valve 120 is decreased by ΔOP₂ (FIG. 2/STEP 126). Accordingly, a flow rate of the target liquid in the second flow path 12 and thus in the target substance removal apparatus 1 is reduced. Subsequently, processing of measuring the first concentration C₁ and the second concentration C₂ (FIG. 2/STEP 012) and thereafter is repeated.

Adjustment may be made for the degree-of-opening increase amount ΔOP₁ of the first flow rate control valve 110 and/or the degree-of-opening decrease amount ΔOP₂ of the second flow rate control valve 120 so that the smaller the rate of decrease (dΔC/dt) of the concentration difference ΔC, the larger the amount.

On the other hand, when the determination result is negative (FIG. 2/STEP 124 . . . NO), processing of measuring the first concentration C₁ and the second concentration C₂ (FIG. 2/STEP 012) and thereafter is repeated.

When the concentration difference ΔC is determined to be included in the designated concentration difference range [ΔCₘᵢₙ, ΔCₘₐₓ] (FIG. 2/STEP 100 . . . 0), a determination of whether or not the first difference index i₀₁ exceeds 0 is made (FIG. 2/STEP 102). When the concentration difference ΔC is determined to be included in the designated concentration difference range [ΔCₘᵢₙ, ΔCₘₐₓ] (FIG. 2/STEP 100 . . . 0), the degree of opening OP₁ of the first flow rate control valve 110 and the degree of opening OP₂ of the second flow rate control valve 120 are maintained as-is.

When the determination result is positive (FIG. 2/STEP 102 . . . YES), the first difference index i₀₁ is reset to “0” (FIG. 2/STEP 104) and, subsequently, a determination is made as to whether or not the second difference index i₀₂ exceeds 0 (FIG. 2/STEP 106). When the determination result is negative (FIG. 2/STEP 102 . . . NO), a determination is made as to whether or not the second difference index i₀₂ exceeds 0 (FIG. 2/STEP 106).

When the determination result is positive (FIG. 2/STEP 106 . . . YES), the second difference index i₀₂ is reset to “0” (FIG. 2/STEP 108) and, subsequently, processing of connector X1 and thereafter is executed. When the determination result is negative (FIG. 2/STEP 106 . . . NO), processing of connector X1 and thereafter is executed as-is.

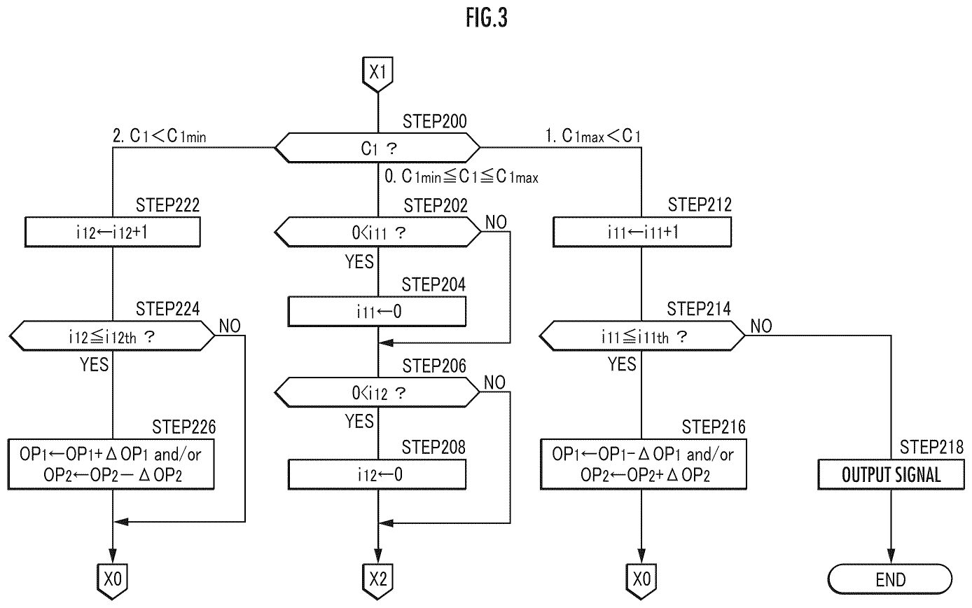

Processing of connector X1 and thereafter will be described with reference to the flowchart in FIG. 3. First, a determination is made as to whether the first concentration C₁ is included in a first designated concentration range [C₁ₘᵢₙ, C₁ₘₐₓ] or is less than a lower limit value C₁ₘᵢₙ or exceeds an upper limit value C₁ₘₐₓ thereof (FIG. 3/STEP 200).

When the first concentration C; is determined to exceed the upper limit value C₁ₘₐₓ of the first designated concentration range (FIG. 3/STEP 200 . . . 1), a first upstream index in is increased by “1” (FIG. 3/STEP 212). The first upstream index “i₁₁” is an index representing a duration of a situation in which the first concentration C₁ exceeds the upper limit value C₁ₘₐₓ of the first designated concentration range. Adjustment may be made for an amount of increase in the first upstream index i₁₁ so that the smaller a rate of decrease (dC₁/dt) of the first concentration C₁ and/or a rate of decrease (dC₂/dt) of the second concentration C₂, the larger the amount (for example, significantly more than “1” such as “2” or “3”). Next, a determination is made as to whether the first upstream index i₁₁ is equal to or lower than a first upstream threshold inn (FIG. 3/STEP 214).

When the determination result is positive (FIG. 3/STEP 214 . . . YES), the operation of the first flow rate control valve 110 and/or the second flow rate control valve 120 is controlled so that the degree of opening OP₁ of the first flow rate control valve 110 is decreased by ΔOP₁ and/or the degree of opening OP₂ of the second flow rate control valve 120 is increased by ΔOP₂ (FIG. 3/STEP 216). Accordingly, a flow rate of the target liquid in the second flow path 12 and thus in the target substance removal apparatus 1 is increased. Then, processing of connector X0 and thereafter is executed. In other words, processing of measuring the first concentration C₁ and the second concentration C₂ (FIG. 2/STEP 012) and thereafter is repeated.

When the first concentration C₁ is determined to exceed the upper limit value C₁ₘₐₓ of the first designated concentration range (FIG. 3/STEP 200 . . . 1), after newly measuring the first concentration C₁, processing of determining whether the new first concentration C₁ is included in the first designated concentration range [C₁ₘᵢₙ, C₁ₘₐₓ] or is less than the lower limit value C₁ₘᵢₙ or exceeds the upper limit value C₁ₘₐₓ thereof (FIG. 3/STEP 200) may be executed.

Adjustment may be made for the degree-of-opening decrease amount ΔOP₁ of the first flow rate control valve 110 and/or the degree-of-opening increase amount ΔOP₂ of the second flow rate control valve 120 so that the smaller the rate of decrease (dC₁/dt) of the first concentration C₁ and/or the rate of decrease (dC₂/dt) of the second concentration C₂, the larger the amount.

On the other hand, when the determination result is negative (FIG. 3/STEP 214 . . . NO), a warning indicating that the target substance removal apparatus 1 must be replaced or the operation of the target liquid treatment apparatus must be stopped is output through an output interface capable of wired communication or wireless communication with the control apparatus 200 in a form that the user can visually and/or audibly recognize (FIG. 3/STEP 218). Subsequently, the series of processing ends. When the determination result is negative (FIG. 3/STEP 214 . . . NO), processing of connector X0 and thereafter may be executed immediately without executing intermediate processing steps.

When the first concentration C₁ is determined to be less than the lower limit value C₁ₘᵢₙ of the first designated concentration range (FIG. 3/STEP 200 . . . 2), a second upstream index i₁₂ is increased by “1” (FIG. 3/STEP 222). The second upstream index “i₁₂” is an index representing a duration of a situation in which the first concentration C₁ is less than the lower limit value C₁ₘᵢₙ of the first designated concentration range. Adjustment may be made for an amount of increase in the second upstream index i₁₂ so that the smaller a rate of increase (dC₁/dt) of the first concentration C₁ and/or a rate of increase (dC₂/dt) of the second concentration C₂, the larger the amount (for example, significantly more than “1” such as “2” or “3”). Next, a determination is made as to whether the second upstream index i₁₂ is equal to or lower than a second upstream threshold i₁₂ₜₕ (FIG. 3/STEP 224).

When the determination result is positive (FIG. 3/STEP 224 . . . YES), the operation of the first flow rate control valve 110 and/or the second flow rate control valve 120 is controlled so that the degree of opening OP₁ of the first flow rate control valve 110 is increased by ΔOP₁ and/or the degree of opening OP₂ of the second flow rate control valve 120 is decreased by ΔOP₂ (FIG. 3/STEP 226). Accordingly, a flow rate of the target liquid in the second flow path 12 and thus in the target substance removal apparatus 1 is reduced. Subsequently, processing of measuring the first concentration C₁ and the second concentration C₂ (FIG. 2/STEP 012) and thereafter is repeated.

When the first concentration C₁ is determined to be less than the lower limit value C₁ₘᵢₙ of the first designated concentration range (FIG. 3/STEP 200 . . . 2), the operation of each of the first flow rate control valve 110 and the second flow rate control valve 120 may be controlled so that the degree of opening OP₁ of the first flow rate control valve 110 is immediately increased to the maximum value OP₁ₘₐₓ (for example, 100%) and the degree of opening OP₂ of the second flow rate control valve 120 is immediately decreased to the minimum value OP₂ₘᵢₙ (for example, 0%). In this case, subsequently, after newly measuring the first concentration Ct, processing of determining whether the new first concentration C₁ is included in the first designated concentration range [C₁ₘᵢₙ, C₁ₘₐₓ] or is less than the lower limit value C₁ₘᵢₙ or exceeds the upper limit value C₁ₘₐₓ thereof (FIG. 3/STEP 200) may be executed.

Adjustment may be made for the degree-of-opening increase amount ΔOP₁ of the first flow rate control valve 110 and/or the degree-of-opening decrease amount ΔOP₂ of the second flow rate control valve 120 so that the smaller the rate of increase (dC₁/dt) of the first concentration C₁ and/or the rate of increase (dC₂/dt) of the second concentration C₂, the larger the amount.

On the other hand, when the determination result is negative (FIG. 3/STEP 224 . . . NO), processing of measuring the first concentration C₁ and the second concentration C₂ (FIG. 2/STEP 012) and thereafter is repeated.

When the first concentration C₁ is determined to be included in the first designated concentration range [C₁ₘᵢₙ, C₁ₘₐₓ] (FIG. 3/STEP 200 . . . 0), a determination of whether or not the first upstream index i₁₁ exceeds 0 is made (FIG. 3/STEP 202). When the first concentration C₁ is determined to be included in the first designated concentration range [C₁ₘᵢₙ, C₁ₘₐₓ] (FIG. 3/STEP 200 . . . 0), the degree of opening OP₁ of the first flow rate control valve 110 and the degree of opening OP₂ of the second flow rate control valve 120 are maintained as-is.

When the determination result is positive (FIG. 3/STEP 202 . . . YES), the first upstream index i₁₁ is reset to “0” (FIG. 3/STEP 204) and, subsequently, a determination is made as to whether or not the second upstream index i₁₂ exceeds 0 (FIG. 3/STEP 206). When the determination result is negative (FIG. 3/STEP 202 . . . NO), a determination is made as to whether or not the second upstream index i₁₂ exceeds 0 (FIG. 3/STEP 206).

When the determination result is positive (FIG. 3/STEP 206 . . . YES), the second upstream index i₁₂ is reset to “0” (FIG. 3/STEP 208) and, subsequently, processing of connector X2 and thereafter is executed. When the determination result is negative (FIG. 3/STEP 206 . . . NO), processing of connector X2 and thereafter is executed as-is.

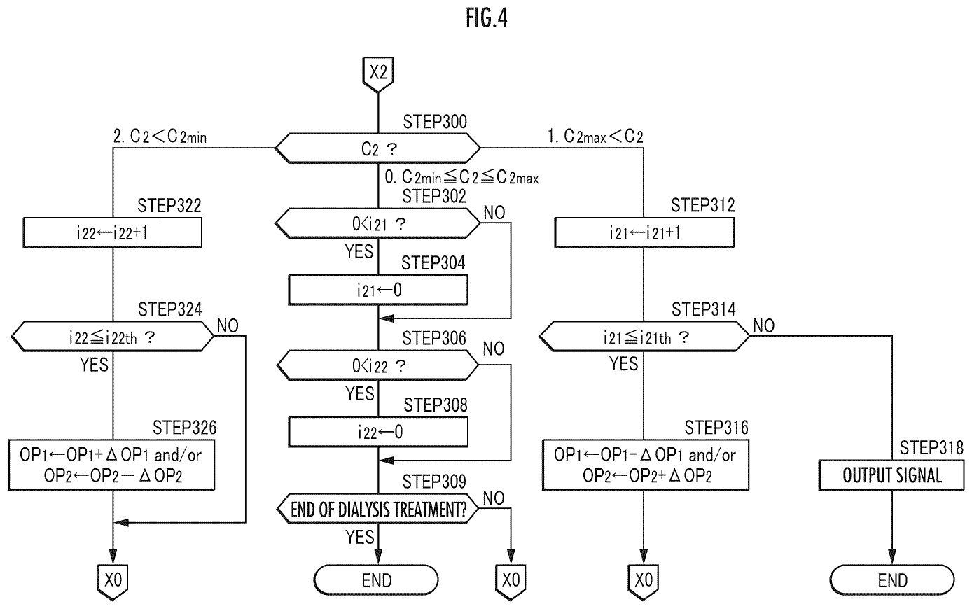

Processing of connector X2 and thereafter will be described with reference to the flowchart in FIG. 4. First, a determination is made as to whether the second concentration C₂ is included in a second designated concentration range [C₂ₘᵢₙ, C₂ₘₐₓ] or is less than a lower limit value C₂ₘᵢₙ or exceeds an upper limit value C₂ₘₐₓ thereof (FIG. 4/STEP 300).

When the second concentration C₂ is determined to exceed the upper limit value C₂ₘₐₓ of the second designated concentration range (FIG. 4/STEP 300 . . . 1), a first downstream index i₂₁ is increased by “1” (FIG. 4/STEP 312). The first downstream index “i₂₁” is an index representing a duration of a situation in which the second concentration C₂ exceeds the upper limit value C₂ₘₐₓ of the second designated concentration range. Adjustment may be made for an amount of increase in the first downstream index i₂₁ so that the smaller a rate of decrease (dC₂/dt) of the second concentration C₂, the larger the amount (for example, significantly more than “1” such as “2” or “3”). Next, a determination is made as to whether the first downstream index i₂₁ is equal to or lower than a first downstream threshold i₂₁ₜₕ (FIG. 4/STEP 314).

When the determination result is positive (FIG. 4/STEP 314 . . . YES), the operation of the first flow rate control valve 110 and/or the second flow rate control valve 120 is controlled so that the degree of opening OP₁ of the first flow rate control valve 110 is decreased by ΔOP₁ and/or the degree of opening OP₂ of the second flow rate control valve 120 is increased by ΔOP₂ (FIG. 4/STEP 316). Accordingly, a flow rate of the target liquid in the second flow path 12 and thus in the target substance removal apparatus 1 is increased. Then, processing of connector X0 and thereafter is executed. In other words, processing of measuring the first concentration C₁ and the second concentration C₂ (FIG. 2/STEP 012) and thereafter is repeated.

When the second concentration C₂ is determined to exceed the upper limit value C₂ₘₐₓ of the second designated concentration range (FIG. 4/STEP 300 . . . 1), processing of connector X0 and thereafter may be executed immediately without executing intermediate processing steps.

Adjustment may be made for the degree-of-opening decrease amount ΔOP₁ of the first flow rate control valve 110 and/or the degree-of-opening increase amount ΔOP₂ of the second flow rate control valve 120 so that the smaller the rate of decrease (dC₂/dt) of the second concentration C₂, the larger the amount.

On the other hand, when the determination result is negative (FIG. 4/STEP 314 . . . NO), a warning indicating that the target substance removal apparatus 1 must be replaced or the operation of the target liquid treatment apparatus must be stopped is output through an output interface capable of wired communication or wireless communication with the control apparatus 200 in a form that the user can visually and/or audibly recognize (FIG. 4/STEP 318). Subsequently, the series of processing ends. When the determination result is negative (FIG. 4/STEP 314 . . . NO), processing of connector X0 and thereafter may be executed immediately without executing intermediate processing steps.

When the second concentration C₂ is determined to be less than the lower limit value C₂ₘᵢₙ of the second designated concentration range (FIG. 4/STEP 300 . . . 2), a second downstream index i₂₂ is increased by “1” (FIG. 4/STEP 322). This is an index representing a duration of a situation in which the second concentration C₂ is less than the lower limit value C₂ₘᵢₙ of the second designated concentration range. Adjustment may be made for an amount of increase in the second downstream index i₂₂ so that the smaller a rate of increase (dC₂/dt) of the second concentration C₂, the larger the amount (for example, significantly more than “1” such as “2” or “3”). Next, a determination is made as to whether the second downstream index i₂₂ is equal to or lower than a second downstream threshold i₂₂ₜₕ (FIG. 4/STEP 324).

When the determination result is positive (FIG. 4/STEP 324 . . . YES), the operation of the first flow rate control valve 110 and/or the second flow rate control valve 120 is controlled so that the degree of opening OP₁ of the first flow rate control valve 110 is increased by ΔOP₁ and/or the degree of opening OP₂ of the second flow rate control valve 120 is decreased by ΔOP₂ (FIG. 4/STEP 326). Accordingly, a flow rate of the target liquid in the second flow path 12 and thus in the target substance removal apparatus 1 is reduced. Subsequently, processing of measuring the first concentration C₁ and the second concentration C₂ (FIG. 2/STEP 012) and thereafter is repeated.

When the second concentration C₂ is determined to be less than the lower limit value C₂ₘᵢₙ of the second designated concentration range (FIG. 4/STEP 300 . . . 2), the operation of each of the first flow rate control valve 110 and the second flow rate control valve 120 may be controlled so that the degree of opening OP₁ of the first flow rate control valve 110 is immediately increased to the maximum value OP₁ₘₐₓ (for example, 100%) and the degree of opening OP₁ of the second flow rate control valve 120 is immediately decreased to the minimum value OP₂ₘᵢₙ (for example, 0%). In this case, subsequently, after newly measuring the second concentration C₂, processing of determining whether the new second concentration C₂ is included in the second designated concentration range [C₂ₘᵢₙ, C₂ₘₐₓ] or is less than the lower limit value C₂ₘᵢₙ or exceeds the upper limit value C₂ₘₐₓ thereof (FIG. 4/STEP 300) may be executed. In addition, when the second concentration C₂ is determined to be included in the second designated concentration range [C₂ₘᵢₙ, C₂ₘₐₓ] (FIG. 4/STEP 300 . . . 0), processing of connector X0 and thereafter may be executed immediately without executing intermediate processing steps.

Adjustment may be made for the degree-of-opening increase amount ΔOP₁ of the first flow rate control valve 110 and/or the degree-of-opening decrease amount ΔOP₂ of the second flow rate control valve 120 so that the smaller the rate of increase (dC₂/dt) of the second concentration C₂, the larger the amount.

On the other hand, when the determination result is negative (FIG. 4/STEP 324 . . . NO), processing of measuring the first concentration C₁ and the second concentration C₂ (FIG. 2/STEP 012) and thereafter is repeated.

When the second concentration C₂ is determined to be included in the second designated concentration range [C₂ₘᵢₙ, C₂ₘₐₓ] (FIG. 4/STEP 300 . . . 0), a determination of whether or not the first downstream index i₂₁ exceeds 0 is made (FIG. 4/STEP 302). When the second concentration C₂ is determined to be included in the second designated concentration range [C₂ₘᵢₙ, C₂ₘₐₓ] (FIG. 4/STEP 300 . . . 0), the degree of opening OP₁ of the first flow rate control valve 110 and the degree of opening OP₂ of the second flow rate control valve 120 are maintained as-is.

When the determination result is positive (FIG. 4/STEP 302 . . . YES), the first downstream index i₂₁ is reset to “0” (FIG. 4/STEP 304) and, subsequently, a determination is made as to whether or not the second downstream index i₂₂ exceeds 0 (FIG. 4/STEP 306). When the determination result is negative (FIG. 4/STEP 302 . . . NO), a determination is made as to whether or not the second downstream index 122 exceeds 0 (FIG. 4/STEP 306).

When the determination result is positive (FIG. 4/STEP 306 . . . YES), the second downstream index i₂₂ is reset to “0” (FIG. 4/STEP 308) and, subsequently, a determination is made as to whether or not end conditions are satisfied (FIG. 4/STEP 309). When the determination result is negative (FIG. 4/STEP 306 . . . NO), a determination is made as-is as to whether or not the end conditions are satisfied (FIG. 4/STEP 309).

For example, the end conditions include a condition that the target liquid treatment apparatus is instructed to stop operation by an OFF operation of the operation switch or the like and/or a condition that a continuous operation time of the target liquid treatment apparatus has exceeded a predetermined time. When a determination is made that the end conditions are not satisfied (FIG. 4/STEP 309 . . . NO), processing of connector X0 and thereafter is executed. In other words, processing of measuring the first concentration C₁ and the second concentration C₂ (FIG. 2/STEP 012) and thereafter is repeated. On the other hand, when a determination is made that the end conditions are satisfied (FIG. 4/STEP 309 . . . YES), the series of processing ends.

### ADVANTAGEOUS EFFECTS

With the target liquid treatment apparatus according to the present invention, from the perspective of removing substances to be removed in a target liquid using the target substance removal apparatus 1, the control apparatus 200 appropriately adjusts the degree of opening OP₁ of the first flow rate control valve 110 and/or the degree of opening OP₂ of the second flow rate control valve 120 based on measurement results of the first concentration C; and the second concentration C₂.

### Other Embodiments of the Present Invention

While a determination is made in the embodiment described above as to whether a concentration difference ΔC is included in a designated concentration difference range [ΔCₘᵢₙ, ΔCₘₐₓ] or is less than a lower limit value ΔCₘᵢₙ or exceeds an upper limit value ΔCₘₐₓ thereof (refer to FIG. 2/STEP 100), as another embodiment, a determination may be made as to whether the concentration difference ΔC is less than a concentration difference threshold (for example, a value corresponding to ΔCₘᵢₙ).

In the other embodiment, when the concentration difference ΔC is determined to be less than the concentration difference threshold, processing of FIG. 2/STEP 106→STEP 108→STEP 112→ . . . STEP 116, and STEP 118 is executed. When the concentration difference ΔC is determined to be equal to or more than the concentration difference threshold, processing of FIG. 2/STEP 102→STEP 104→STEP 122→ . . . STEP 126 is executed. The connector X0 following FIG. 2/STEP 126 may be replaced with the connector X1.

While a determination is made in the embodiment described above as to whether the first concentration C₁ is included in a first designated concentration range [C₁ₘᵢₙ, C₁ₘₐₓ] or is less than a lower limit value C₁ₘᵢₙ or exceeds an upper limit value C₁ₘₐₓ thereof (refer to FIG. 3/STEP 200), as another embodiment, a determination may be made as to whether the first concentration C₁ is less than a first concentration threshold (for example, a value corresponding to C₁ₘᵢₙ).

In the other embodiment, when the first concentration C₁ is determined to exceed the first concentration threshold, processing of FIG. 3/STEP 206→STEP 208→STEP 212→ . . . STEP 216, and STEP 218 is executed. When the first concentration C₁ is determined to be equal to or less than the first concentration threshold, processing of FIG. 3/STEP 202→STEP 204→STEP 222→ . . . STEP 226 may be executed and the connector X0 following FIG. 3/STEP 226 may be replaced with the connector X2.

While a determination is made in the embodiment described above as to whether the second concentration C₂ is included in a second designated concentration range [C₂ₘᵢₙ, C₂ₘₐₓ] or is less than a lower limit value C₂ₘᵢₙ of exceeds an upper limit value C₂ₘₐₓ thereof (refer to FIG. 4/STEP 300), as another embodiment, a determination may be made as to whether the second concentration C₂ is less than a second concentration threshold (for example, a value corresponding to C₂ₘᵢₙ).

In the other embodiment, when the second concentration C₂ is determined to exceed the second concentration threshold, processing of FIG. 4/STEP 306→STEP 308→STEP 312→ . . . STEP 316, and STEP 318 is executed. When the second concentration C₂ is determined to be equal to or less than the second concentration threshold, processing of FIG. 4/STEP 302→STEP 304→STEP 322→ . . . STEP 326 is executed.

While after processing of FIG. 2/STEP 100 and thereafter, processing of connector X1 and thereafter (processing of FIG. 3/STEP 200 and thereafter) is executed and, furthermore, processing of connector X2 and thereafter (processing of FIG. 4/STEP 300 and thereafter) is executed in the embodiment described above, as another embodiment, an order of the three series of processing may be interchanged, only one series of processing among the three series of processing may be executed, or two series of processing among the three series of processing may be executed in any order.

An adjustment amount ΔOP₁ of the degree of opening OP₁ of the first flow rate control valve 110 in each of FIG. 2/STEP 116 and STEP 126, FIG. 3/STEP 216 and STEP 226, and FIG. 4/STEP 316 and STEP 326 may be the same or may mutually differ (such as an increase amount ΔOP₁ being smaller than a decrease amount ΔOP₁). In a similar manner, an adjustment amount ΔOP₂ of the degree of opening OP₂ of the second flow rate control valve 120 in each of FIG. 2/STEP 116 and STEP 126, FIG. 3/STEP 216 and STEP 226, and FIG. 4/STEP 316 and STEP 326 may be the same or may mutually differ (such as an increase amount ΔOP₂ being larger than a decrease amount ΔOP₂).

Reference Signs List

| | |
|---|---|
| 1 | target substance removal apparatus |
| 10 | ion adsorbent |
| 11 | first flow path |
| 12 | second flow path |
| 100 | housing |
| 101 | inflow port |
| 102 | outflow port |
| 110 | first flow rate control valve |
| 112 | first check valve |
| 120 | second flow rate control valve |
| 122 | second check valve |
| 200 | control apparatus |
| S₁ | first concentration sensor |
| S₂ | second concentration sensor |

1. A target liquid treatment apparatus, comprising:
a first flow path through which a dialysis effluent, a hemodiafiltrate, or a hemodiafiltration fluid flowing out of a dialyzer or a diafilter flows as a target liquid;
a second flow path which joins the first flow path after branching from the first flow path;
a target substance removal apparatus which is provided in the second flow path and which is capable of selectively removing a substance to be removed contained in the target liquid;
a first flow rate control valve which is provided in the first flow path between a branching point and a joining point of the second flow path;
a second flow rate control valve which is provided in the second flow path;
a first concentration sensor which measures a concentration of the substance to be removed in the target liquid as a first concentration on an upstream side of the joining point of the second flow path in the first flow path;
a second concentration sensor which measures a concentration of the substance to be removed in the target liquid as a second concentration on a downstream side of the joining point of the second flow path in the first flow path; and
a control apparatus for adjusting respective degrees of opening of the first flow rate control valve and the second flow rate control valve based on measured results of the first concentration and the second concentration, wherein
the control apparatus is configured to
execute, when a difference between the first concentration and the second concentration is less than a concentration difference threshold, at least one of decreasing the degree of opening of the first flow rate control valve and increasing the degree of opening of the second flow rate control valve.

2. The target liquid treatment apparatus according to claim 1, wherein
the control apparatus is configured to
execute, when the difference between the first concentration and the second concentration is less than a lower limit value of a designated concentration difference range as the concentration difference threshold, at least one of decreasing the degree of opening of the first flow rate control valve and increasing the degree of opening of the second flow rate control valve, and
execute, when the difference between the first concentration and the second concentration exceeds an upper limit value of the designated concentration difference range, at least one of increasing the degree of opening of the first flow rate control valve and decreasing the degree of opening of the second flow rate control valve.

3. The target liquid treatment apparatus according to claim 1, wherein
the control apparatus is configured to
control, when the difference between the first concentration and the second concentration is less than the concentration difference threshold, an operation of at least one of the first flow rate control valve and the second flow rate control valve so that the lower a rate of decrease of the difference the higher at least one of a decrease rate of the degree of opening of the first flow rate control valve and an increase rate of the degree of opening of the second flow rate control valve.

4. The target liquid treatment apparatus according to claim 2, wherein
the control apparatus is configured to
control, when the difference between the first concentration and the second concentration exceeds the upper limit value of the designated concentration difference range, an operation of at least one of the first flow rate control valve and the second flow rate control valve so that the higher a rate of increase of the difference, the higher at least one of an increase rate of the degree of opening of the first flow rate control valve and a decrease rate of the degree of opening of the second flow rate control valve.

5. The target liquid treatment apparatus according to claim 1, wherein
the control apparatus is configured to
execute, when the first concentration exceeds an upper limit value of a first designated concentration range as a first concentration threshold, at least one of decreasing the degree of opening of the first flow rate control valve and increasing the degree of opening of the second flow rate control valve, and
execute, when the first concentration is less than the first concentration threshold, at least one of increasing the degree of opening of the first flow rate control valve and decreasing the degree of opening of the second flow rate control valve.

6. The target liquid treatment apparatus according to claim 1, wherein
the control apparatus is configured to
control, when the first concentration exceeds a first concentration threshold, an operation of at least one of the first flow rate control valve and the second flow rate control valve so that the higher a rate of increase of the first concentration, the higher at least one of a decrease rate of the degree of opening of the first flow rate control valve and an increase rate of the degree of opening of the second flow rate control valve.

7. The target liquid treatment apparatus according to claim 5, wherein
the control apparatus is configured to
control, when the first concentration is less than a lower limit value of the first designated concentration range, an operation of at least one of the first flow rate control valve and the second flow rate control valve so that the higher a rate of decrease of the first concentration, the higher at least one of an increase rate of the degree of opening of the first flow rate control valve and a decrease rate of the degree of opening of the second flow rate control valve.

8. The target liquid treatment apparatus according to claim 1, wherein
the control apparatus is configured to
execute, when the second concentration exceeds an upper limit value of a second designated concentration range as a second concentration threshold, at least one of decreasing the degree of opening of the first flow rate control valve and increasing the degree of opening of the second flow rate control valve, and
execute, when the second concentration is less than the second concentration threshold, at least one of increasing the degree of opening of the first flow rate control valve and decreasing the degree of opening of the second flow rate control valve.

9. The target liquid treatment apparatus according to claim 1, wherein
the control apparatus is configured to
control, when the second concentration exceeds a second concentration threshold, an operation of at least one of the first flow rate control valve and the second flow rate control valve so that the higher a rate of increase of the second concentration, the higher at least one of a decrease rate of the degree of opening of the first flow rate control valve and an increase rate of the degree of opening of the second flow rate control valve.

10. The target liquid treatment apparatus according to claim 8, wherein
the control apparatus is configured to
control, when the second concentration is less than a lower limit value of the second designated concentration range, an operation of at least one of the first flow rate control valve and the second flow rate control valve so that the higher a rate of decrease of the second concentration, the higher at least one of an increase rate of the degree of opening of the first flow rate control valve and a decrease rate of the degree of opening of the second flow rate control valve.
